(19) United States
(12) Patent Application Publication
(10) Pub. No.: US 2005/0100531 A1
(43) Pub. Date: May 12, 2005

(54) Probiotic therapies

(76) Inventor: John Bienenstock, Toronto (CA)

Correspondence Address: JACOBSON HOLMAN;PROFESSIONAL LIMITED LIABILITY COMPANY, 400 SEVENTH STREET, N.W., WASHINGTON, DC, 20004, US

(21) Appl. No.: 11/008,232

(22) Filed: Dec. 10, 2004

Related U.S. Application Data
(63) Continuation of application No. PCT/IE03/00090, filed on Jun. 13, 2003.

(30) Foreign Application Priority Data
Jun. 13, 2002 (IE) 2002/0479

(51) Int. Cl. A61K 48/00; C12N 1/21

(52) U.S. Cl. 424/93.2; 514/44; 435/252.3

(57) ABSTRACT

Use of a bacterial strain or an active derivative, fragment or mutant thereof which selectively upregulates the production of nerve growth factor (NGF), brain derived neurotrophic factor (BDNF), neurotrophin 3 (NT3) or neurotrophin 4 (NT4) in the treatment and/or prophylaxis of various disorders especially inflammatory disorders. The bacterial strain may be a Lactobacillus especially,Lactobacillus reuteri.

### INTRODUCTION

The invention relates to the immunoregulatory and therapeutic effects of a bacterial strain in particular immunoregulatory and therapeutic effects of a bacterial strain. The defence mechanisms to protect the human gastrointestinal tract from colonization by intestinal bacteria are highly complex and involve both immunological and non-immunological aspects (V. J. McCracken and H. R. Gaskins, ‘Probiotics a critical review’, Horizon Scientific Press, UK, 1999, p. 278.). Innate defence mechanisms include the low pH of the stomach, bile salts, peristalsis, mucin layers and anti-microbial compounds such as lysozyme (D. C. Savage, ‘Microbial Ecology of the Gut’, Academic Press, London, 1997, p.278.). Immunological mechanisms include specialized lymphoid aggregates, underlying M cells, called peyers patches, which are distributed throughout the small intestine and colon (M. F. Kagnoff. Gastroenterol. 1993, 105, 1275). Luminal antigens presented at these sites result in stimulation of appropriate T and B cell subsets with establishment of cytokine networks and secretion of antibodies into the gastrointestinal tract (M. R. Neutra and J-P Kraebenbuhl, ‘Essentials of mucosal immunology’, Academic Press, San Diego, 1996, p.29., M. E. Lamm. Ann. Rev. Microbiol. 1997, 51, 311). In addition, antigen presentation may occur via epithelial cells to intraepithelial lymphocytes and to the underlying lamina propria immune cells (S. Raychaudhuri et al. Nat Biotechnol., 1998, 16, 1025). Therefore, the host invests substantially in immunological defence of the gastrointestinal tract. However, as the gastrointestinal mucosa is the largest surface at which the host interacts with the external environment, specific control mechanisms must be in place to regulate immune responsiveness to the 100 tons of food, which is handled by the gastrointestinal tract over an average lifetime (F. Shanahan, ‘Physiology of the gastrointestinal tract’, Raven Press, 1994, p.643.). Furthermore, the gut is colonised by over 500 species of bacteria numbering 10¹¹-10¹²/g in the colon. Thus, these control mechanisms must be capable of distinguishing non-pathogenic adherent bacteria from invasive pathogens, which would cause significant damage to the host. In fact, the intestinal flora contributes to defence of the host by competing with newly ingested potentially pathogenic micro-organisms.

The microflora on mucosal surfaces are vast in number and complexity. Many hundreds of bacterial strains exist and account for approximately 90% of the cells found in the human body, the remainder of the cells being human. The vast majority of these bacterial strains do not cause disease and may actually provide the host with significant health benefits (e.g. bifidobacteria and lactobacilli). These bacterial strains are termed commensal organisms. Mechanism(s) exist whereby the immune system at mucosal surfaces can recognise commensal non-pathogenic flora as being different to pathogenic organisms.

Bacteria present in the human gastrointestinal tract can promote inflammation. Aberrant immune responses to the indigenous microflora have been implicated in certain disease states, such as inflammatory bowel disease (Brandzeag P. et al. Springer Semin. Immunopathol., 1997, 18, 555). Antigens associated with the normal flora usually lead to immunological tolerance and failure to achieve this tolerance is a major mechanism of mucosal inflammation (Stallmach A. et al., Immunol. Today, 1998, 19, 438). Evidence for this breakdown in tolerance includes an increase in antibody levels directed against the gut flora in patients with inflammatory bowel disease (IBD).

A method of inhibiting the production of undesirable molecules such as pro-inflammatory molecules in vivo would have significant therapeutic potential.

### STATEMENTS OF INVENTION

According to the invention there is provided use of a bacterial strain or an active derivative, fragment or mutant thereof which selectively upregulates the production of nerve growth factor (NGF), brain derived neurotrophic factor (BDNF), neurotrophin 3 (NT3) or neurotrophin 4 (NT4) in the treatment and/or prophylaxis of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris.

Preferably the bacterial strain is derived from the human commensal flora which stimulate the production of nerve growth factor (NGF), brain derived neurotrophic factor (BDNF), neurotrophin 3 (NT3) or neurotrophin 4 (NT4).

In one embodiment of the invention the bacterial strain is a Lactobacillus. Preferably the Lactobacillus is Lactobacillus reuteri.

The bacteria may be viable or non viable. Also included are components or mutants of the strain.

The invention provides use of a bacterial strain or an active derivative, fragment or mutant thereof which selectively upregulates the production of NGF, BDNF, NT3 or NT4 in the treatment and/or prophylaxis of various diseases such as inflammatory diseases.

The invention further provides use of NGF, BDNF, NT3 or NT4 or an active derivative, fragment or mutant thereof in the treatment and/or prophylaxis of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris.

The invention also provides use of NGF, BDNF, NT3 or NT4 or an active derivative, fragment or mutant thereof in the treatment and/or prophylaxis of various diseases such as inflammatory diseases.

The invention further provides use of a bacterial strain or an active derivative, fragment or mutant thereof which selectively upregulates the production of NGF, BDNF, NT3 or NT4 and IL-10 in the treatment and/or prophylaxis of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris.

The invention also provides a method for screening the therapeutic potential of bacterial strains for use in disease specific therapies comprising stimulating epithelial cells with different bacterial species to induce different cytokine profiles.

The invention further provides a method for screening the therapeutic potential of bacterial strains for use in disease specific therapies comprising stimulating epithelial cells with different bacterial species to selectively upregulate NGF, BDNF, NT3 or NT4 and induce different cytokine profiles.

The invention also provides a formulation comprising a bacterial strain or an active derivative, fragment or mutant thereof which induces NGF, BDNF, NT3 or NT4.

The invention further provides a formulation comprising a bacterial strain or an active derivative, fragment or mutant thereof which induces NGF, BDNF, NT3 or NT4 and/or IL-10.

The invention also provides a formulation comprising a bacterial strain or an active derivative, fragment or mutant thereof which induces NGF, BDNF, NT3 or NT4 for use in the prevention and/or treatment of various diseases such as inflammatory diseases.

The invention also provides a formulation comprising NGF, BDNF, NT3 or NT4 or an active derivative, fragment or mutant thereof for use in the prevention and/or treatment of various diseases such as inflammatory diseases.

The invention further provides a vaccine comprising a bacterial strain or an active derivative, fragment or mutant thereof which selectively upregulates the production of NGF, BDNF, NT3 or NT4 in the treatment and/or prophylaxis of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris.

The invention also provides a vaccine comprising a bacterial strain or an active derivative, fragment or mutant thereof which selectively upregulates the production of NGF, BDNF, NT3 or NT4 in the treatment and/or prophylaxis of various diseases such as inflammatory diseases.

The invention also provides a vaccine comprising NGF, BDNF, NT3 or NT4 or an active derivative, fragment or mutant thereof in the treatment and/or prophylaxis of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, eachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris.

Throughout the description the terrn derivative is taken to include active forms of the bacterial strain with modifications which do not substantially effect the activity of the strain. The term mutant is taken to include strains with amino acid variations which do not substantially effect the activity of the strain. The term fragment is taken to include sub-units encoded by a nucleic acid sequence present in all or part of the parent bacterial strain.

### BRIEF DESCRIPTION OF THE DRAWINGS

FIG. 1 is a bar graph showing the induction of IL-10 by NGF in a dose dependant manner;

FIG. 2 is a bar graph showing the induction of NGF by IL-10 in a dose dependant manner;

FIG. 3 is a bar graph showing the temporal induction of NGF by IL-10;

FIG. 4 is a bar graph showing the stimulatory effect of a lactobacillus species on NGF production;

FIG. 5 is a bar graph showing the inhibitory effect of a lactobacillus strain on TNFα induced IL-8 mRNA levels; and

FIG. 6 is a bar graph showing the inhibitory effect of a lactobacillus strain on TNFα induced IL-8 protein levels.

### DETAILED DESCRIPTION

One of the mechanisms whereby probiotic organisms may protect against mucosal inflammation may be directly or indirectly through interaction with the mucosal epithelium thereby causing the epithelium to upregulate and express molecules which are anti-inflammatory. These would include cytokines such as IL-10, neurotrophins such as nerve growth factor (NGF), brain derived neurotrophic factor (BDNF), neurotrophin 3 (NT3) or neurotrophin 4 (NT4) and products of arachidonic acid such as PGE₂.

The present invention is directed toward bacterial strains, which have immuno-regulatory and therapeutic effects due to their stimulation of NGF activity. It is also envisaged that the bacterial strains, would have immuno-regulatory and therapeutic effects due to their stimulation of BDNF, NT3 or NT4 activity.

It was found in the present invention that particular strains of commensal micro-organisms such as Bifidobacteria and Lactobacilli, induced NGF synthesis resulting in immunomodulatory effects in vitro.

Following exposure to a commensal micro-organism epithelial cells induced NGF production which resulted in inhibition of pro-inflammatory cytokine generation. NGF and the cytokines appear to reciprocally upregulate each other.

It was surprisingly found in the present invention that on stimulation of epithelial cells with Lactobacillus reuteri NGF was selectively induced resulting in the attenuation of TNF induced IL-8 cytokine production.

Lactobacillus is commercially available. Lactobacillus reuteri strain type RF 14249 and RF 20013 is deposited under the designation F275 (DSM20016), JCM112, ATCC23272, NCD02589.

Nerve growth factor (NGF), for which discovery Professor Rita Levi-Montalcini received the Nobel Prize, was the first neurotrophin, or nerve growth factor to be discovered. It is essential for the growth of nerves in the peripheral nervous system, the autonomic nervous system, especially the sympathetic, and many components of the central nervous system. It is synthesised and secreted by a large number of cells in addition to those of the nervous system: these include structural cells such as the glia, and fibroblasts, cells of the immune system such as T-lymphocytes (especially TH2) and cells involved in the inflammatory process such as eosinophils, mast cells and dendritic cells. It has pleiotropic functions in addition to its multiple effects in the nervous system. While it is synthesised in larger form, the active component is a 2.5S molecule with a molecular weight of about 26K. It's chromosomal location is 1p13. It promotes the survival and growth of neurons but in addition has many effects in many different systems which include promotion of human hemopoietic colony growth of basophils and eosinophils, the promotion of synthesis by B-cells of IgG4 and the prevention of apoptosis in neutrophils, eosinophils and mast cells (Bienenstock et al., 2000). It has also been shown to have wound healing properties (Matsuda et al., 1998) and appears to protect against a mucosal inflammatory model of hapten-induced colitis (Reinshagen et al., 2000). Most recently, T-cells transfected with the gene for NGF have been shown to protect the central nervous system against damage in an autoimmune model of demyelinating disease, autoimmune encephalomyelitis (Flugel et al., 2001).

NGF is an important neurotrophin and is essential for many different physiological functions. The induction of NGF results in improved immunological control and promotes wound healing. A method of inducing NGF production with the attenuation of different cytokines has therefore large therapeutic potential.

Other important neurotrophins essential for many different physiological functions are brain derived neurotrophic factor (BDNF), neurotrophin 3 (NT3) or neurotrophin 4 (NT4). These neurotrophins are closely related to NGF and it is envisaged that these neurotrophins would be induced by bacterial strains in a similar manner as NGF is induced.

The invention has potential therapeutic value in the prophylaxis and/or treatment of dysregulated immunological control, such as undesirable inflammatory reactions (e.g. inflammatory bowel disease).

It is envisaged however that stimulation of epithelial cells with different bacterial species would result in epithelial cells with different cytokine profiles. These different immuno-therapeutic properties may therefore be applicable to a wide range of disease states.

Other Bifidobacteria and Lactobacilli strains potentially have the same effect as L. reuteri. Such Bifidobacteria and Lactobacilli are typically isolated from the microbial flora within the human gastrointestinal tract. The immune system within the gastrointestinal tract cannot have a pronounced reaction to members of this flora, as the resulting inflammatory activity would also destroy host cells and tissue function. Therefore, some mechanism(s) exist whereby the immune system can recognise commensal non-pathogenic members of the gastrointestinal flora as being different to pathogenic organisms. This ensures that damage to host tissues is restricted and a defensive barrier is still maintained.

The strain may be a genetically modified mutant or it may be a naturally occurring variant thereof.

Preferably the strain is in the form of viable cells. Alternatively the strain may be in the form of non-viable cells.

The invention also relates to the potential of bacterial strains in customising epithelial cell phenotype and function. In this way customisation of disease specific therapies may be accomplished using a selection of bacterial strains.

The effects on cytokine production are specific for each of the probiotic strains examined. Thus specific probiotic strains may be selected for normalising an exclusive cytokine imbalance particular for a specific disease type. Customisation of disease specific therapies can be accomplished using a selection of probiotic strains.

Recognition of bacterial species by epithelial cells results in distinct patterns of cytokine production and immune responses. The cytokines produced by epithelial cells are secreted into the extracellular milieu. These cytokines deliver an informative signal to neighbouring cells, which do not necessarily have to be in physical contact with the epithelial cell. This “bystander” effect results in many different cell types being influenced by the cytokine network established by bacterial stimulated epithelial cells.

Immune Education

The enteric flora is important to the development and proper function of the intestinal immune system. In the absence of an enteric flora, the intestinal immune system is underdeveloped, as demonstrated in germ free animal models, and certain functional parameters are diminished, such as macrophage phagocytic ability and immunoglobulin production (Crabbe et al., 1968, Wostmann et al., 1996). The importance of the gut flora in stimulating non-damaging immune responses is becoming more evident. The increase in incidence and severity of allergies in the western world has been linked with an increase in hygiene and sanitation, concomitant with a decrease in the number and range of infectious challenges encountered by the host. This lack of immune stimulation may allow the host to react to non-pathogenic, but antigenic, agents resulting in allergy or autoimmunity. Deliberate consumption of a series of non-pathogenic immunomodulatory bacteria would provide the host with the necessary and appropriate educational stimuli for proper development and control of immune function.

The human immune system plays a significant role in the aetiology and pathology of a vast range of human diseases. Hyper and hypo-immune responsiveness results in, or is a component of, the majority of disease states. One family of biological entities, termed cytokines, are particularly important to the control of immune processes. Pertubances of these delicate cytokine networks are being increasingly associated with many diseases. These diseases include but are not limited to inflammatory disorders, immunodeficiency, inflammatory bowel disease, irritable bowel syndrome, cancer (particularly those of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and acne vulgaris.

As the majority of cytokines may have both pro and anti-inflammatory activities, patterns or networks of cytokine release have been associated with different types of immune responses. The existence of T cells, which differ in their pattern of cytokine secretion, allows differentiation of inflammatory or immune responses into at least three categories, cell mediated or humoral responses or Th3/Tr1 regulatory responses. Th1 responses are categorised by IFNα, TNFβ and IL-2 production leading to a cell-mediated response while Th2 cells secrete IL-4, IL-5, IL-9, IL-10 and IL-13 resulting in a humoral response. Th3/Tr1 responses are characterised by T cell secretion of the regulatory cytokines IL-10 and TGF. Differentiation of T cells into either network depends on the cytokine milieu in which the original antigen priming occurs (Seder et al., 1992). In addition, the polarisation of T cell subpopulations are influenced by a number of other cell types including dendritic cells and epithelial cells. (Mosmann & Sad, 1996). Certain types of stimulation may also direct this response, such as immune complex deposition within inflammatory sites which increases IL-6 and IL-10 production and inhibits production of TNFα and IL-1β thus influencing the Th1/Th2 balance. For successful elimination of pathogens, the correct cytokine network needs to be established. For example, the intracellular bacterium Listeria monocytogenes elicits a Th1 response while the extracellular parasite Nippostrongylus brasiliensis requires a Th2 response. Each of these T cell subsets produce cytokines that are autocrine growth factors for that subset and promote differentiation of naive T cells into that subset (Trinchieri et al., 1996). These two subsets also produce cytokines that cross-regulate each other's development and activity. IFNγ amplifies Th1 development and inhibits proliferation of Th2 T cells while IL-10 blocks Th1 activation. Tr1 cells have a profound suppressive effect on antigen-specific T cell responses mediated by secretion of IL-10 and TGFβ (Groux et al., 1997) and cytokine independent mechanisms such as direct cell-cell contact.

The cytokine networks involved in immune responses are subject to a complex number of control pathways that normally result in restriction of cellular damage and eradication of the infectious organism. However, unregulated release of these cytokines can have damaging consequences. Incorrect Th1/Th2 responses contribute to the pathogenesis of certain diseases. For instance, the healing form of leprosy (tuberculoid lesion) is associated with a Th1 response while uncontrolled leprosy (lepromatous lesion) is associated with a Th2 response. Chronic inflammatory responses can lead to the death of the host. For instance, rats infected with the protozoan parasite Trypanosoma brucei become cachectic, develop anaemia and eventually die. Production of proinflammatory cytokines has been associated with the pathogenesis of many disorders. In Langerhans cell histiocytosis, cytokines may be involved in some of the tissue damage seen with this disease (Kannourakis & Abbas, 1994). Rheumatoid arthritis is a chronic inflammatory disease of the synovial joints resulting in cartilage destruction and bone erosion (Kouskoff et al., 1996). Elevated levels of proinflammatory cytokines have been detected from patients with rheumatoid arthritis and these levels could be associated with disease activity, altered energy metabolism and food intake (Roubenoff et al., 1994). In patients with sepsis, cardiovascular shock and organ dysfunction may be initiated by the production of proinflammatory cytokines stimulated by the infectious organism particularly in patients with cerebral malaria (Kwiatkowski et al., 1990). Certain alleles of polymorphic sites associated with TNFα production have been shown to predict patients with cerebral malaria (McGuire et al., 1994) and severe sepsis (Stuber et al., 1996) who will be most adversely affected. Genetic predisposition to increased TNFα production may also be associated with the development of autoimmune diseases such as diabetes and systemic lupus erythematosus. Inhibition of proinflammatory cytokine production has reduced the damage caused by many disease states. IL-1RA reduces the severity of diseases such as shock, lethal sepsis, inflammatory bowel disease, experimental arthritis and proliferation of human leukaemic cells (for review see Dinarello, 1992). Inhibition of TNFα in septic shock prevents the syndrome of shock and tissue injury despite persistent bacteraemia in animal models. Loss of the TNF receptor type 1 in knock-out mice protects against endotoxic shock (Pfeiffer et al., 1993). Anti-cytokine strategies in humans with sepsis have yielded disappointing results possibly due to complications such as the late administration of these factors after the initial inflammatory insult. However, studies involving neutralising TNFα antibodies in rheumatoid arthritis and Crohn's disease have had considerable success with significant reductions in disease activity being observed (Moreland et al., 1997, Stack et al., 1997). Inhibition of transcription factors, such as NF-κB, which are responsible for intracellular signalling in the inflammatory response have been successful in reducing tissue damage in animals with chronic intestinal inflammation (Neurath et al., 1996). Moreover, adoptive transfer of T cells secreting IL-10 inhibited colitis in a murine model (Asserman et al., 1999). In addition, consumption of certain bacterial strains results in attenuation of gastrointestinal inflammatory activity (O'Mahony et al., 2001, Rembacken et al., 1999). Therefore, while the inflammatory response is critical to the defence and repair of host tissues, uncontrolled responses can result in significant tissue and organ damage and may result in the death of the host.

TGFβ refers to a family of closely related molecules termed TGFβ1 to -β5 (Roberts & Sporn, 1990). All are released from cells in a biologically inactive form due to their association with a latency protein which is believed to be a critical regulatory step. Three receptors have been identified for TGFβ. Only two of these receptors transduce an intracellular signal suggesting a decoy function for the third receptor. Like the MIP family, TGFβ also functions as a chemotactic factor for both monocytes and neutrophils. However, this cytokine has diverse effects as both pro and anti-inflammatory effects have been described. Aggregated platelets following vascular injury release TGFβ resulting in inflammatory cell recruitment to the tissue. Activated monocytes and neutrophils synthesise TGFβ further increasing cellular recruitment. Monocyte integrin expression is also enhanced by TGFβ as is the induction of collagenase type IV which may aid movement through basement membranes into inflamed sites (Wahl et al., 1993). TGFβ increases the expression of FcγRIII (CD16) which recognises antibody bound cells thereby increasing phagocytic activity. The production of inflammatory cytokines by monocytes can also be stimulated by TGFβ. However, expression of IL-1 receptor antagonist (IL-1RA) is also increased suggesting that this cascade, in part, may be self regulating. TGFβ is also important as a negative regulatory agent. It antagonises the effects of many inflammatory cytokines and inhibits the proliferation of thymocytes, B cells and haemapoietic stem cells. The activity of a number of cell types can be suppressed by TGFβ including natural killer (NK) cells, cytotoxic T lymphocytes and lymphokine activated killer (LAK) cells. TGFβ also has suppressive effects on the release of reactive oxygen and nitrogen intermediates by tissue macrophages (Ding et al., 1990). The immune inhibitory effects of TGFβ can most clearly be observed in its effects on diseases such as experimental arthritis, multiple sclerosis and graft rejection. Through the stimulation of matrix protein production, TGFβ may be important to wound healing which is also indicated by its chemotactic activity for fibroblasts (Roberts & Sporn, 1990). Therefore TGFβ may have important functions with regard to resolution of the inflammatory response and promotion of healing within the inflammatory lesion.

IL-10 is produced by T cells, B cells, monocytes and macrophages (De Waal Malefyt et al., 1991). This cytokine augments the proliferation and differentiation of B cells into antibody secreting cells (Go et al., 1990). IL-10 exhibits mostly anti-inflammatory activities. It up-regulates IL-1RA expression by monocytes and suppresses the majority of monocyte inflammatory activities. IL-10 inhibits monocyte production of cytokines, reactive oxygen and nitrogen intermediates, MHC class II expression, parasite killing and IL-10 production via a feed back mechanism (De Waal Malefyt et al., 1991). This cytokine has also been shown to block monocyte production of intestinal collagenase and type IV collagenase by interfering with a PGE₂-cAMP dependant pathway (Mertz et al., 1994) and therefore may be an important regulator of the connective tissue destruction seen in chronic inflammatory diseases.

Interleukin-8 (IL-8) is one of the cytokines comprising the Macrophage Inflammatory protein family (MIP). The MIP-1 and -2 families represent a group of proteins which are chemotactic factors for leukocytes and fibroblasts. This family of proteins are also called intercrines, as cells other than macrophages are capable of synthesising them. These cells include T and B cells, epithelial cells, fibroblasts, endothelial cells, keratinocytes, smooth muscle cells, synovial cells, neutrophils, chondrocytes, hepatocytes, platelets and tumour cells. MIP-1α, -1β, connective tissue activating protein (CTAP), platelet factor 4 (PF4) and IL-8 stimulate neutrophil chemotaxis. Monocyte chemotactic protein (MCP-1) and RANTES are chemotactic for monocytes while PF4 and CTAP are chemotactic for fibroblasts. Stimulation of epithelial cells by certain pathogenic bacteria or proinflammatory cytokines, such as TNF, results in the release of IL-8 which recruits neutrophils and lymphocytes to damaged or inflamed sites. Roles other than chemotaxis have been described for some of these family members. MCP-1 stimulates monocyte cytostatic activity and superoxide anion release. CTAP and PF4 increase fibroblast proliferation, IL-8 increases vascular permeability while MIP-1α and -1β are pyrogenic. IL-8 is intimately involved in inflammatory responses within the gastrointestinal tract. Stimulation of IL-8 (and other proinflammatory cytokines) contributes to the development of gastrointestinal lesions.

TNFα is a proinflammatory cytokine, which mediates many of the local and systemic effects seen during an inflammatory response. This cytokine is primarily a monocyte or macrophage derived product but other cell types including lymphocytes, neutrophils, NK cells, mast cells, astrocytes, epithelial cells (Neale et al., 1995) endothelial cells and smooth muscle cells can also synthesise TNFα. TNFα is synthesised as a prohormone and following processing the mature 17.5 kDa species can be observed. Purified TNFα has been observed as dimers, trimers and pentamers with the trimeric form postulated to be the active form in vivo. Three receptors have been identified for TNFα. A soluble receptor seems to function as a TNFα inhibitor while two membrane bound forms have been identified with molecular sizes of 60 and 80 kDa respectively (Schall et al., 1990). Local TNFα production at inflammatory sites can be induced with endotoxin and the glucocorticoid dexamethasone inhibits cytokine production. TNFα production results in the stimulation of many cell types. Significant anti-viral effects could be observed in TNFα treated cell lines and the IFNs synergise with TNFα enhancing this effect (Wong & Goeddel, 1986). Endothelial cells stimulated by TNFα produce procoagulant activity, expression of adhesion molecules, IL-1, hematopoitic growth factors, platelet activating factor (PAF) and arachidonic acid metabolites. TNFα stimulates neutrophil adherence, phagocytosis, degranulation, reactive oxygen intermediate production and may influence cellular migration (Livingston et al., 1989). Leucocyte synthesis of GM-CSF, TGFβ, IL-1, IL-6, PGE₂ and TNFα itself can all be stimulated upon TNFα, administration (Cicco et al., 1990). Programmed cell death (apoptosis) can be delayed in monocytes (Mangan et al., 1991) while effects on fibroblasts include the promotion of chemotaxis and IL-6, PGE₂ and collagenase synthesis. While local TNFα production promotes wound healing and immune responses, the dis-regulated systemic release of TNFα can be severely toxic with effects such as cachexia, fever and acute phase protein production being observed (Dinarello et al., 1988).

Inflammation

Inflammation is the term used to describe the local accumulation of fluid, plasma proteins and white blood cells at a site that has sustained physical damage, infection or where there is an ongoing immune response. Control of the inflammatory response is exerted on a number of levels (for review see Henderson B., and Wilson M. 1998. In “Bacteria-Cytokine interactions in health and disease. Portland Press, 79-130). The controlling factors include cytokines, hormones (e.g. hydrocortisone), prostaglandins, reactive intermediates and leukotrienes. Cytokines are low molecular weight biologically active proteins that are involved in the generation and control of immunological and inflammatory responses, while also regulating development, tissue repair and haematopoiesis. They provide a means of communication between leukocytes themselves and also with other cell types. Most cytokines are pleiotrophic and express multiple biologically overlapping activities. Cytokine cascades and networks control the inflammatory response rather than the action of a particular cytokine on a particular cell type (Arai K I, et al., Annu Rev Biochem 1990;59:783-836). Waning of the inflammatory response results in lower concentrations of the appropriate activating signals and other inflammatory mediators leading to the cessation of the inflammatory response. TNFα is a pivotal proinflammatory cytokine as it initiates a cascade of cytokines and biological effects resulting in the inflammatory state. Therefore, agents which inhibit TNFα are currently being used for the treatment of inflammatory diseases, e.g. infliximab.

Pro-inflammatory cytokines are thought to play a major role in the pathogenesis of many inflammatory diseases, including inflammatory bowel disease (IBD). Current therapies for treating IBD are aimed at reducing the levels of these pro-inflammatory cytokines, including IL-8 and TNFα. Such therapies may also play a significant role in the treatment of systemic inflammatory diseases such as rheumatoid arthritis.

The strains of the present invention may have potential application in the treatment of a range of inflammatory diseases, particularly if used in combination with other anti-inflammatory therapies, such as non-steroid anti-inflammatory drugs (NSAIDs) or Infliximab.

It is unknown whether the complete bacterial cell is required to exert an immuno-modulatory effect or if individual active components of the bacterial strains can be utilised alone. Proinflammatory components of certain bacterial strains have been identified. The proinflammatory effects of gram-negative bacteria are mediated by a number of cellular structures including lipopolysaccharide (LPS). LPS alone induces a proinflammatory network. It is assumed that components of probiotic bacteria possess anti-inflammatory activity, due to the effects of the whole cells. Upon isolation of these components, pharmaceutical grade manipulation is anticipated. Therefore the term bacterial strain as used in this specification refers to active components thereof.

The general use of the bacterial strains is in the form of viable cells. However, it can also be extended to non-viable cells such as killed cultures or compositions containing beneficial factors expressed by the bacterial strains. This could include micro-organisms killed by exposure to altered pH or subjection to pressure. With non-viable cells product preparation is simpler, cells may be incorporated easily into pharmaceuticals and storage requirements are not as limited. Lactobacillus casei YIT 9018 offers an example of the effective use of heat killed cells as a method for the treatment and/or prevention of tumour growth as described in U.S. Pat. No. 4,347,240.

Prebiotics

The introduction of probiotic organisms is accomplished by the ingestion of the micro-organism in a suitable carrier. It would be advantageous to provide a medium that would promote the growth of these probiotic strains in the large bowel. The addition of one or more oligosaccharides, polysaccharides, or other prebiotics enhances the growth of lactic acid bacteria in the gastrointestinal tract (Gibson, G R. Br. J. Nutr. 1998;80 (4):S209-12). Prebiotics refers to any non-viable food component that is specifically fermented in the colon by indigenous bacteria thought to be of positive value, e.g. bifidobacteria, lactobacilli. Types of prebiotics may include those that contain fructose, xylose, soya, galactose, glucose and mannose. The combined administration of a probiotic strain with one or more prebiotic compounds may enhance the growth of the administered probiotic in vivo resulting in a more pronounced health benefit, and is termed synbiotic.

Other Active Ingredients

It will be appreciated that the probiotic strains may be administered prophylactically or as a method of treatment either on its own or with other probiotic and/or prebiotic materials as described above. In addition, the bacteria may be used as part of a prophylactic or treatment regime using other active materials such as those used for treating inflammation or other disorders especially those with an immunological involvement. Such combinations may be administered in a single formulation or as separate formulations administered at the same or different times and using the same or different routes of administration.

The invention will be more clearly understood from the following examples.

### EXAMPLE 1

### NGF Stimulation of Epithelial Cells

Human colonic epithelial cell lines T-84 and Hr-29 were co-incubated with 0, 10, 100 or 1000 ng/ml NGF. IL-6, IL-8, IL-10, TGFβ and NGF mRNA levels were quantified using RT-PCR. NGF increased IL-10 levels in a dose dependant manner (FIG. 1). Maximal induction of IL-10 was observed within one hour of stimulation. None of the other cytokines were induced by NGF stimulation.

### EXAMPLE 2

### IL-10 Stimulation of Epithelial Cells

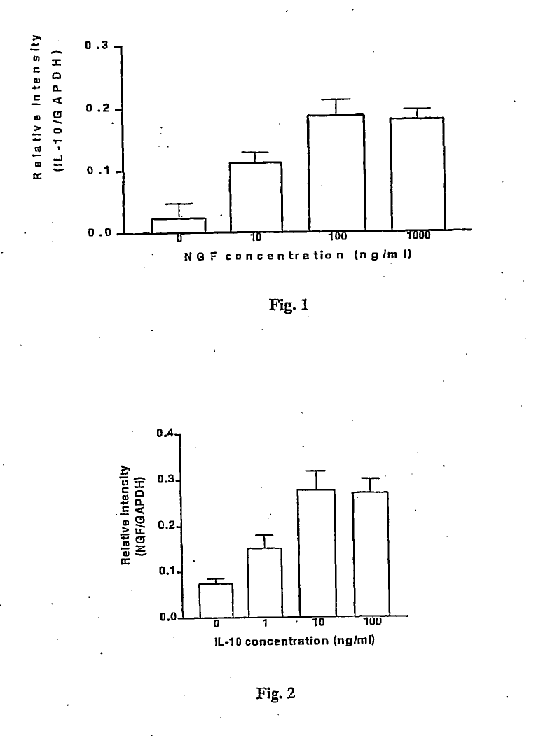

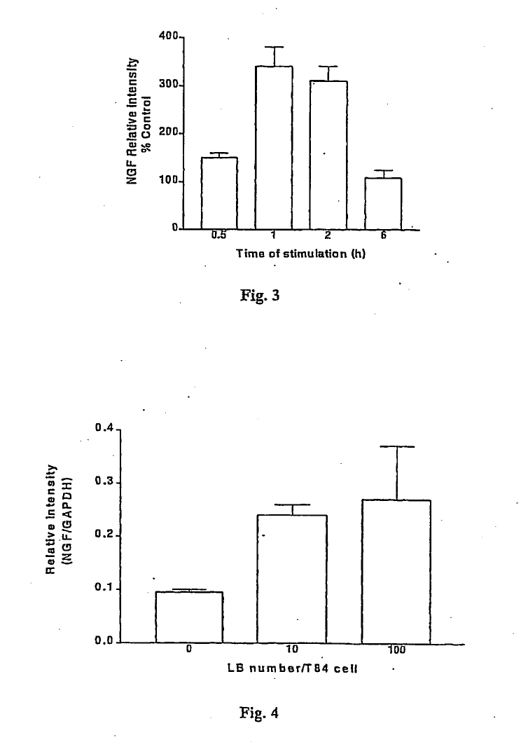

Human colonic epithelial cell lines T-84 and HT-29 were co-incubated with 0, 1, 10 or 100 ng/ml IL-10. IL-6, IL-8, IL-10, TGFβ and NGF mRNA levels were quantified using RT-PCR. IL-10 selectively increased NGF levels in a dose dependant manner (FIG. 2). Maximal induction of NTF was noted following one hour of stimulation (FIG. 3). None of the other cytokines were induced by IL-10 stimulation.

### EXAMPLE 3

### Lactobacilli Selectively Upregulate NGF

Human colonic epithelial cell lines T-84 and HT-29 were co-incubated with Lactobacillus reuteri for 2 hours. IL-6, IL-8, IL-10, and NGF mRNA levels were quantified using RT-PCR. L. reuteri selectively increased NGF levels (FIG. 4). None of the other cytokines were induced by bacterial stimulation.

### EXAMPLE 4

### Lactobacilli Inhibit TNFα Induced IL-8

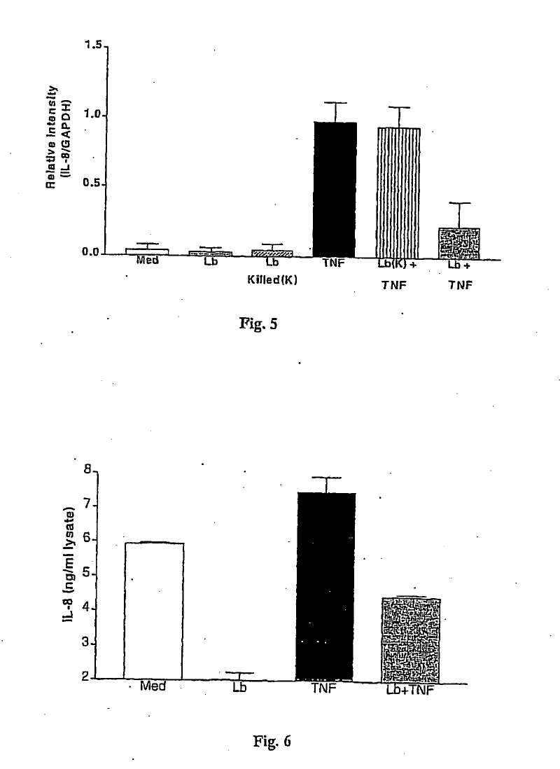

Human colonic epithelial cell lines T-84 and HT-29 were co-incubated with Lactobacillus reuteri for 2 hours, followed by 30 minutes incubation with 10 ng/ml TNFα. IL-8 mRNA levels were quantified using RT-PCR. In addition, intracellular IL-8 levels were measured following co-incubation with brefeldin A for 3 hours. Cells were then lysed and IL-8 levels quantified by ELISA. L. reuteri attenuated TNF induced IL-8 production both at the mRNA and protein level (FIGS. 5 & 6). Heat inactivated bacterial cells did not maintain this immunomodulatory effect (FIG. 5).

The invention is not limited to the embodiments herein before described which may be varied in detail.

### References

Wilmes-Riesenberg M. R, Bearson B., Foster J. W. & Curtiss R. Role of the acid tolerance response in virulence of Salmonella typhimurium. Infect Immun., 1996:1085-92.
Seder R A, Paul W E, Davis M M, Fazekas de St Groth B. The presence of interleukin 4 during in vitro priming determines the lymphokine-producing potential of CD4+ T cells from T cell receptor transgenic mice. J Exp Med 1992 Oct 1;176(4):1091-8.
Mosmann T. R. & Sad S. The expanding universe of T-cell subsets: Th1, Th2 and more. Immunol. Today, 1996; 17:138-46.
Trinchieri G, Peritt D, Gerosa F. Acute induction and priming for cytokine production in lymphocytes. Cytokine Growth Factor Rev 1996 Aug;7(2):123-32.
Maldonado-Lopez R. De Smedt T., Michel P., Godfroid J., Pajak B., Heirman C., Thielemans K., Leo O., Urbain J. & Moser M. CD8 + and CD8− subclasses of dendritic cells direct the development of distinct T helper cells in vivo. J. Exp. Med., 1999; 189:587-92.
Groux H., O'Garra A., Bigler M., Rouleau M., Antonenko S., de Vries J. E. & Roncarolo M. G. A CD4+ T-cell subset inhibits antigen-specific T-cell responses and prevents colitis. Nature, 1997; 389:737-42.
Jonuleit H., Schmitt E., Schuler G., Knop J. & EnK A. H. Induction of interleukin 10-producing, nonproliferating CD4+ T cells with regulatory properties by repetitive stimulation with allogeneic immature human dendritic cells. J. Exp. Med., 2000; 192:1213-22.
Kannourakis G, Abbas A. The role of cytokines in the pathogenesis of Langerhans cell histiocytosis. Br J Cancer Suppl 1994 Sep;23:S37-40.
Kouskoff V, Korganow A S, Duchatelle V, Degott C, Benoist C, Mathis D. Organ-specific disease provoked by systemic autoimmunity. Cell 1996 Nov 29;87(5):811-22.
Roubenoff R, Roubenoff R A, Cannon J G, Kehayias J J, Zhuang H, Dawson-Hughes B, Dinarello C A, Rosenberg IH. Rheumatoid cachexia: cytokine-driven hypermetabolism accompanying reduced body cell mass in chronic inflammation. J Clin Invest 1994 Jun;93(6):2379-86.
Kwiatkowski D, Hill A V, Sambou I, Twumasi P, Castracane J, Manogue KR, Cerami A, Brewster D R, Greenwood B M. TNF concentration in fatal cerebral, non-fatal cerebral, and uncomplicated Plasmodium falciparum malaria. Lancet 1990 Nov 17;336(8725):1201-4.
McGuire W, Hill A V, Allsopp C E, Greenwood B M, Kwiatkowski D. Variation in the TNF-alpha promoter region associated with susceptibility to cerebral malaria Nature 1994 Oct 6;371(6497):508-10.
Stuber F, Petersen M, Bokelmann F, Schade U. A genomic polymorphism within the tumor necrosis factor locus influences plasma tumor necrosis factor-alpha concentrations and outcome of patients with severe sepsis. Crit Care Med 1996 Mar;24(3):381-4.
Dinarello C A. The role of interleukin-1 in host responses to infectious diseases. Infect Agents Dis 1992 Oct;1(5):227-36.
Pfeffer A, Matsuyama T, Kundig T M, Wakeham A, Kishihara K, Shahinian A, Wiegmann K, Ohashi P S, Kronke M, Mak T W. Mice deficient for the 55 kd tumor necrosis factor receptor are resistant to endotoxic shock, yet succumb to L. monocytogenes infection. Cell 1993 May 7;73(3):457-67.
Moreland L W, Baumgartner S W, Schiff M H, Tindall E A, Fleischmann R M, Weaver A L, Ettlinger R E,Cohen S, Koopman W J, Mohler K, Widmer M B, Blosch C M. Treatment of rheumatoid arthritis with a recombinant human tumor necrosis factor receptor (p75)-Fc fusion protein. N Engl J Med 1997 Jul 17;337(3):141-7.
Stack W A, Mann S D, Roy A J, Heath P, Sopwith M, Freeman J, Holmes G, Long R, Forbes A, Kamm M A. Randomised controlled trial of CDP571 antibody to tumour necrosis factor-alpha in Crohn's disease. Lancet 1997 Feb 22;349(9051):521-4.
Neurath M F, Pettersson S, Meyer zum Buschenfelde K H, Strober W. Local administration of antisense phosphorothioate oligonucleotides to the p65 subunit of NF-cappa B abrogates established experimental colitis in mice. Nat Med 1996 Sep;2(9):998-1004.
Asserman C., Mauze S., Leach M. W., Coffman R. L. & Powrie F. An essential role for interleukin 10 in the function of regulatory T cells that inhibit intestinal inflammation. J. Exp. Med., 1999; 190:995-1003.
O'Mahony L, Feeney M, O'Halloran S, Murphy L, Kiely B, Fitzgibbon J, Lee G, O'Sullivan G, Shanahan F. Collins J K. Probiotic impact on microbial flora, inflammation and tumour development in IL-10 knockout mice. Alim. Pharmacol. Ther.2001; 15(8):1219-1225.
Rembacken B J, Snelling A M, Hawkey P M, Chalmers D M, Axon A T. Non-pathogenic Escherichia coli versus mesalazine for the treatment of ulcerative colitis: a randomised trial. Lancet 1999; 354: 635-9.
Bienenstock J. Mast cell-nerve interactions: possible significance of nerve growth factor. In Mast Cells and Basophils in Physiology, Pathology and Host Defense, ed. Marone G. Lichtenstein L M, Galli S J, Academic Press, 2000.
Matsuda H, Koyama H. Sato H, et al. Role of nerve growth factor in cutaneous wound healing: accelerating effects in normal and healing-impaired diabetic mice. J Exp Med 1998; 187:297-306.
Reinshagen M, Rohm H, Steinkamp M, et al. Protective role of neurotrophins in experimental inflammation of the rat gut Gastroenterology 2000; 119:368-376.
Flugel A, Matsumuro K, Neumann H, et al. Anti-inflammatory activity of nerve growth factor in experimental autoimmune encephalomyelitis: inhibition of monocyte transendothelial migration. Eur J Immunol 2001; 31:11-22.
Roberts A B, Flanders K C, Heine UI, Jakowlew S, Kondaiah P, Kim S J, Sporn M B. Transforming growth factor-beta: multifunctional regulator of differentiation and development. Philos Trans R Soc Lond B Biol Sci 1990 Mar 12;327(1239):145-54.
Wahl S M, Allen J B, Weeks B S, Wong H L, Klotmann P E. Transforming growth factor beta enhances integrin expression and type IV collagenase secretion in human monocytes. Proc Natl Acad Sci U S A 1993 May 15;90(10):4577-81.
Ding A, Nathan C F, Graycar J, Derynck R, Stuehr D J, Srimal S. Macrophage deactivating factor and transforming growth factors-beta 1-beta 2and -beta 3 inhibit induction of macrophage nitrogen oxide synthesis by IFN-gamma. J Immunol 1990 Aug 1; 145(3):940-4. Arai I C I, Lee F, Miyajima A, Miyatake S, Arai N, Yokota T. Cytokines: coordinators of immune and inflammatory responses. Annu Rev Biochem 1990;59:783-836.
Aiello F B, Longo D L, Overton R, Takacs L, Durum S K. A role for cytokines in antigen presentation: IL-1 and IL-4 induce accessory functions of antigen-presenting cells. J Immunol 1990 Apr 1;144(7):2572-81.
Corcoran M L, Stetler-Stevenson W G, Brown P D, Wahl L M. Interleukin 4 inhibition of prostaglandin E2 synthesis blocks interstitial collagenase and 92-kDa type IV collagenase/gelatinase production by human monocytes. J Biol Chem 1992 Jan 5;267(1):515-9.
Mangan D F, Welch G R, Wahl S M. Lipopolysaccharide, tumor necrosis factor-alpha, and IL-1 beta prevent programmed cell death (apoptosis) in human peripheral blood monocytes. J Immunol 1991 Mar 1;146(5):1541-6.
Hart P H, Ahern M J, Smith M D, Finlay-Jones J J. Regulatory effects of IL-13 on synovial fluid macrophages and blood monocytes from patients with inflammatory arthritis. Clin Exp Immunol 1995 Mar;99(3):331-7.
de Waal Malefyt R, Haanen J, Spits H, Roncarolo M G, te Velde A, Figdor C, Johnson K, Kastelein R, Yssel H. de Vries J E. Interleukin 10 (IL-10) and viral IL-10 strongly reduce antigen-specific human T cell proliferation by diminishing the antigen-presenting capacity of monocytes via downregulation of class II major histocompatability complex expression. J Exp Med 1991 Oct 1;174(4):915-24.
Go N F, Castle B E, Barrett R, Kastelein R, Dang W, Mosmann T R, Moore K W, Howard M. Interleukin 10, a novel B cell stimulatory factor: unresponsiveness of X chromosome-linked immunodeficiency B cells. J Exp Med 1990 Dec 1;172(6):1625-31.
Mertz P M, DeWitt D L, Stetler-Stevenson W G, Wahl L M. Interleukin 10 suppression of monocyte prostaglandin H synthase-2. Mechanism of inhibition of prostaglandin-dependent matrix metalloproteinase production. J Biol Chem 1994 Aug 19;269(33):21322-9. Ma X, Aste-Amezaga M, Gri G, Gerosa F, Triochieri G. Immunomodulatory functions and molecular regulation of IL-12. Chem Immunol 1997;68:1-22.
Schmitt E, Rude E, Germann T. The immunostimulatory function of IL-12 in T-helper cell development and its regulation by TGF-beta, IFN-gamma and IL-4. Chem Immunol 1997;68:70-85.
D'Andrea A, Ma X, Aste-Amezaga M, Paganin C, Trinchieri G. Stimulatory and inhibitory effects of interleukin (IL)-4 and IL-13 on the production of cytokines by human peripheral blood mononuclear cells: priming for IL-12 and tumor necrosis factor alpha production. J Exp Med 1995 Feb 1;181(2):537-46.
Leonard J P, Waldburger K E, Schaub R G, Smith T. Hewson AK, Cuzner M L, Goldman S J. Regulation of the inflammatory response in animal models of multiple sclerosis by interteukin-12. Crit Rev Immunol 1997;17(5-6):545-53.
Donnelly R P, Fenton M J, Finbloom D S, Gerrard T L. Differential regulation of IL-1 production in human monocytes by IFN-gamma and IL-4. J Immunol 1990 Jul 15;145(2):569-75.
Wahl L M, Corcoran M E, Mergenhagen S E, Finbloom D S. Inibition of phospholipase activity in human monocytes by IFN-gamma blacks endogenous prostaglandin E2-dependent collagenase production. J Immunol 1990 May 1;144(9):3518-22.
Neale T J, Ruger B M, Macaulay H, Dunbar P R, Hasan Q, Bourke A, Murray-McIntosh R P, Kitching A R. Tumor necrosis factor-alpha is expressed by glomerular visceral epithelial cells in human membranous nephropathy. Am J Pathol 1995 Jun;146(6):1444-54.
Schall T J, Lewis M, Koller K J, Lee A, Rice G C, Wong G H, Gatanaga T, Granger G A, Lentz R, Raab H. et al. Molecular cloning and expression of a receptor for human tumor necrosis factor. Cell 1990 Apr 20;61(2):361-70.
Wong G H, Goeddel D V. Tumour necrosis factors alpha and beta inhibit virus replication and synergise with interferons. Nature 1986 Oct 30-Nov 5;323(6091):819-22.
Livingston D H, Appel S H, Sonnenfeld G, Malangoni M A. The effect of tumor necrosis factor-alpha and interferon-gamma on neutrophil function. J Surg Res 1989 Apr;46(4):322-6.
Cicco N A, Lindemann A, Content J, Vandenbussche P, Lubbert M, Gauss J, Mertelsmann R, Herrmann F. Inducible production of interleukin-6 by human polymorphonuclear neutrophils: role of granulocyte-macrophage colony-stimulating factor and tumor necrosis factor-alpha. Blood 1990 May 15;75(10):2049-52.
Mangan D F, Welch G R, Wahl S M. Lipopolysaccharide, tumor necrosis factor-alpha, and IL-1 beta prevent programmed cell death (apoptosis) in human peripheral blood monocytes. J Immunol 1991 Mar 1;146(5):1541-6.
Dinarello C A, Cannon J G, Wolff S M. New concepts on the pathogenesis of fever. Rev Infect Dis 1988 Jan-Feb;10(1):168-89.

1. A method for the treatment and/or prophylaxis of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmiune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris comprising use of a bacterial strain or an active derivative, fragment or mutant thereof which selectively upregulates the production of nerve growth factor (NGF), brain derived neurotrophic factor (BDNF), neurotrophin 3 (NT3) or neurotrophin 4 (NT4).

2. A method as claimed in claim 1 wherein the bacterial strain is derived from the human commensal flora which stimulate the production of nerve growth factor (NGF), brain derived neurotrophic factor (BDNF), neurotrophin 3 (NT3) or neurotrophin 4 (NT4).

3. A method as claimed in claim 1 wherein the bacterial strain is a Lactobacillus.

4. A method as claimed in claim 3 wherein the Lactobacillus is a Lactobacillus reuteri.

5. A method as claimed in claim 1 wherein the bacteria are non viable or components or mutants thereof.

6. A method for the treatment and/or prophylaxis of various diseases such as inflammatory diseases comprising use of a bacterial strain or an active derivative, fragment or mutant thereof which selectively upregulates the production of NGF.

7. A method for the treatment and/or prophylaxis of inflammatory disorders, immunodeficiency, inflammatory bowel disease, irritable bowel syndrome, cancer particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris comprising use of nerve growth factor (NGF), brain derived neurotrophic factor (BDNF), neurotrophin 3 (NT3) or neurotrophin 4 (NT4) or an active derivative, fragment or mutant thereof.

8. A method for the treatment and/or prophylaxis of various diseases such as inflammatory diseases comprising use of Nerve Growth Factor (NGF) or an active derivative, fragment or mutant thereof.

9. A method for the treatment and/or prophylaxis of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris comprising use of a bacterial strain or an active derivative, fragment or mutant thereof which selectively upregulates the production of NGF, BDNF, NT3 or NT4 and IL-10.

10. A method for screening the therapeutic potential of bacterial strains for use in disease specific therapies comprising stimulating epithelial cells with different bacterial species to induce different cytokine profiles.

11. A method for screening the therapeutic potential of bacterial strains for use in disease specific therapies comprising stimulating epithelial cells with different bacterial species to selectively upregulate NGF, BDNF, NT3 or NT4 and induce different cytokine profiles.

12. A formulation comprising a bacterial strain or an active derivative, fragment or mutant thereof which induces NGF, BDNF, NT3 or NT4.

13. A formulation comprising a bacterial strain or an active derivative, fragment or mutant thereof which induces NGF, BDNF, NT3 or NT4 and/or IL-10.

14. A formulation comprising a bacterial strain or an active derivative, fragment or mutant thereof which induces NGF, BDNF, NT3 or NT4 for use in the prevention and/or treatment of various diseases such as inflammatory diseases.

15. A formulation comprising NGF, BDNF, NT3 or NT4 or an active derivative, fragment or mutant thereof for use in the prevention and/or treatment of various diseases such as inflammatory diseases.

16. A vaccine comprising a bacterial strain or an active derivative, fragment or mutant thereof which selectively upregulates the production of NGF, BDNF, NT3 or NT4 in the treatment and/or prophylaxis of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischemia, nutritional disorders, osteoporosis, endocrine disorders, epidermal disorders, psoriasis and/or acne vulgaris.

17. A vaccine comprising a bacterial strain or an active derivative, fragment or mutant thereof which selectively upregulates the production of NGF, BDNF, NT3 or NT4 in the treatment and/or prophylaxis of various diseases such as inflammatory diseases.

18. A vaccine comprising NGF, BDNF, NT3 or NT4 or an active derivative, fragment or mutant thereof in the treatment and/or prophylaxis of inflammatory disorders, immunodeficiency, inflammatory bowel disease, irritable bowel syndrome, cancer (particularly of the gastrointestinal and immune systems), diarrhoeal disease, antibiotic associated diarrhoea, paediatric diarrhoea, appendicitis, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhoea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anaemia, disorders of the blood coagulation system, renal disease, disorders of the central nervous system, hepatic disease, ischaemia, nutritional disorders, osteoporosis, endocrine disorders, epidermal disorders, psoriasis and/or acne vulgaris.
